SEQUENCE LISTING (8 sequences)

SEQ ID NO: 1 - DNA, 50 bp, synthetic construct
  misc_feature 1..50: note = Primer forward Igk
  source 1..50: mol_type = other DNA
gcagtcgaacatgtagctgactcaggtcactgctcatcagatggcgggaa

SEQ ID NO: 2 - DNA, 50 bp, synthetic construct
  misc_feature 1..50: note = Primer reverse Igk
  source 1..50: mol_type = other DNA
tggatcacttgtgcaagcatcacatcgtagaagagcttcaacaggggaga

SEQ ID NO: 3 - DNA, 50 bp, synthetic construct
  misc_feature 1..50: note = Primer forward Ig lambda
  source 1..50: mol_type = other DNA
gcagtcgaacatgtagctgactcaggtcacagtgtggccttgttggcttg

SEQ ID NO: 4 - DNA, 50 bp, synthetic construct
  misc_feature 1..50: note = Primer reverse Ig lambda
  source 1..50: mol_type = other DNA
tggatcacttgtgcaagcatcacatcgtaggtcacgcatgaagggagcac

SEQ ID NO: 5 - DNA, 50 bp, synthetic construct
  misc_feature 1..50: note = Primer forward Ig gamma
  source 1..50: mol_type = other DNA
gcagtcgaacatgtagctgactcaggtcacaccggttcggggaagtagtc

SEQ ID NO: 6 - DNA, 50 bp, synthetic construct
  misc_feature 1..50: note = Primer reverse Ig gamma
  source 1..50: mol_type = other DNA
tggatcacttgtgcaagcatcacatcgtagcttctcatgctccgtgatgc

SEQ ID NO: 7 - DNA, 51 bp, synthetic construct
  misc_feature 1..51: note = Primer forward Ig alpha
  source 1..51: mol_type = other DNA
gcagtcgaacatgtagctgactcaggtcacggctcctgggggaagaagccc

SEQ ID NO: 8 - DNA, 51 bp, synthetic construct
  misc_feature 1..51: note = Primer reverse Ig alpha
  source 1..51: mol_type = other DNA
tggatcacttgtgcaagcatcacatcgtagccttctcctgcatggtgggcc

(19) United States
(12) Patent Application Publication
(10) Pub. No.: US 2025/0092438 A1
(43) Pub. Date: Mar. 20, 2025

(54) METHOD FOR THE IDENTIFICATION OF THE WHOLE SEQUENCE OF THE VARIABLE REGION OF THE HEAVY AND LIGHT CHAINS OF IMMUNOGLOBULINS

(71) Applicants: UNIVERSITA’ DEGLI STUDI DI PAVIA, Pavia (IT); FONDAZIONE IRCCS POLICLINICO SAN MATTEO, Pavia (IT)

(72) Inventors: Mario Ulisse NUVOLONE, Pavia (IT); Giovanni PALLADINI, Pavia (IT); Pasquale CASCINO, Novara (IT)

(21) Appl. No.: 18/580,990

(22) PCT Filed: Jul. 20, 2022
(86) PCT No.: PCT/IB2022/056700

§ 371 Date: Jan. 19, 2024

(30) Foreign Application Priority Data
Jul. 21, 2021 (IT) 102021000019334

(51) Int. Cl. C12Q 1/6804; C12Q 1/6806

(52) U.S. Cl. CPC C12Q 1/6804; C12Q 1/6806

(57) ABSTRACT

The present invention relates to a method for the identification of the whole nucleotide sequence of the variable region of the heavy and or light chains of immunoglobulins in a biological sample and the quantification of their relative frequency. The invention is particularly used for the identification of monoclonal heavy and light chains, i.e. tumours, in biological samples from patients suffering from a monoclonal gammapathy.

The present invention relates to a method for the identification of the whole nucleotide sequence of the variable region of the heavy and/or light chains of immunoglobulins in a biological sample and the quantification of their relative frequency. The invention is particularly used for the identification of monoclonal heavy and light chains, i.e. tumours, in biological samples from patients suffering from a monoclonal gammapathy.

Monoclonal gammopathies, including multiple myeloma, Waldenström's macroglobulinemia, monoclonal gammapathies of clinical significance (MGCS), and the presymptomatic stage called monoclonal gammapathy of undetermined significance (MGUS), occur when a B lymphocyte or plasma cell, which produces a specific antibody, undergoes a tumour transformation process, which leads to the production of a population of identical cells (i.e. the lymphocyte or plasma cell clone), which all produce the same antibody (i.e. the monoclonal antibody or monoclonal component). Each patient has a unique monoclonal component, whose sequence can be used as a tumour fingerprint for tracking the presence of the lymphocyte or plasma cell clone.

In MGCS, the underlying lymphocyte or plasma cell clone is usually small and poorly proliferating; patients however develop potentially fatal organ damage that is governed by the specific sequence of the patient's light/heavy immunoglobulin chain.

The prototype of MGCS is systemic amyloidosis from immunoglobulin light chains (AL amyloidosis), wherein a small plasma cell (or sometimes lymphocyte) clone secretes an unstable immunoglobulin light chain, which undergoes a pathological process of three-dimensional misfolding (the so-called misfolding process), forming extracellular systemic deposits of amyloid fibrils, exerting cytotoxicity, subverting tissue architecture, and ultimately causing potentially fatal (multi)-organ dysfunction.

The sequencing of the monoclonal component from a large number of patients could therefore allow a deepening of the knowledge, currently limited, of the molecular mechanisms underlying these diseases.

Furthermore, a knowledge of the sequence of the monoclonal component in individual patients could improve personalized medicine approaches, such as the search, with highly sensitive methods, for residual tumour cells after therapy in the context of diagnostic evaluations collectively referred to as the study of the minimal residual disease (or measurable residual disease).

The specific nucleotide sequence that encodes a given immunoglobulin heavy or light chain is the result of a combinatorial process—called V (D)J recombination—and a mutational process—called somatic hypermutation—which affects fragments of specific genes during the development of B lymphocytes and plasma cells deriving from them.

The sequencing of heavy and light chains of immunoglobulins is technically hampered by the lack of an a priori knowledge of which gene fragments were used and by the fact that relevant biological samples (e.g., bone marrow or peripheral blood) typically contain a large number of B lymphocytes/plasma cells that produce different immunoglobulins, with different sequences.

Methods for the sequencing of the variable region of light or heavy chains of immunoglobulins in cells deriving from bone marrow have been described in literature, aimed at identifying the clonal sequence in patients with a lymphocyte or plasma cell clone by reverse PCR, cloning, bacterial transformation and Sanger sequencing. [1-5]. In particular, one method employs reverse PCR effected in a single step, with DNA polymerase without a proof-reading activity, followed by cloning, bacterial transformation, and Sanger sequencing of single bacterial colonies [1]. In the single-step reverse PCR phase, the method exploits the fact that the Taq polymerase used is not provided with a proof-reading activity and that the enzyme incorporates an additional A in 3′ at the end of the DNA synthesis reaction. The amplicon obtained, in fact, with the additional A at the 3′ ends of the two strands, is subsequently subjected to cloning according to the TOPO TA system (which allows the ligation of the additional As at the 3′ ends of the amplicon with the additional Ts in 5′ of the construct for cloning). The use of a DNA polymerase without a proof-reading activity, necessary for allowing subsequent cloning with the TOPO TA system, is however associated with a greater risk of incorporating a wrong nucleotide during PCR than in cases in which a DNA polymerase with a proof-reading, and therefore more accurate activity, is used. The amplicon obtained after reverse PCR, characterized by the presence of the additional As at the two 3′ ends of the duplex DNA strands, is ligated within a pCR plasmid, containing additional Ts at both 5′ ends. The ligation product is then used for transforming a competent E. coli strain, in which single competent bacterial cells incorporate the plasmid used for the transformation and amplify it by means of a replicative apparatus that is not without errors [6]. After transformation, the bacteria are plated under selective conditions, resistant bacterial colonies are selected, grown and subsequently lysed in order to obtain plasmid DNA. The plasmid DNA thus obtained is digested with EcoRI and the digestion products are examined by means of agarose gel electrophoresis. The plasmids that give rise to a digestion pattern compatible with the successful incorporation of the reverse PCR amplicon are then analyzed by Sanger sequencing, using an antisense NotI oligonucleotide as forward primer and the CLA (for the λ light chain) or CH1 primer (for the heavy chain γ) as a reverse primer. The chromatogram obtained with the forward primer and the reverse primer for each sample are compared to obtain the consensus sequence.

The sequences obtained with the Sanger method are analyzed by EMBL-GeneBank, VBASE and IMGT [1,7-8]. The comparison of the sequences obtained from different bacterial colonies transformed with the amplicon obtained from a given patient allows the identification of a “predominant identical sequence”, which is considered as the sequence of the monoclonal component [1]. There are no precise indications about the minimum number of sequences to be analyzed, nor definitions of the predominance in this context. In studies in which this method was used, an average of 5 sequences per patient were analyzed (range: 3-12 total sequences obtained) and the predominant sequence was the correspondence of 4sequences on average per patient (range: 3-9 corresponding sequences) [7].

In a modified version of the method reported in [1], the reverse PCR in a single step is effected using a high fidelity DNA polymerase with a proof-reading activity and the amplicon obtained, devoid of the additional As at the ends in 3′, is cloned by blunt cloning into a plasmid vector, to then be used for bacterial transformation and subsequent sequencing of single colonies [2].

It should be noted how the cloning and amplification practices of DNA molecules by means of the replicative apparatus of bacterial cells and the subsequent Sanger sequencing, even more so if preceded by an initial amplification by PCR using DNA polymerase without a proof-reading activity, can be associated with the generation of DNA mutations, consequently obtaining artifactual sequences, not present in the initial biological sample to be analyzed, and the result of the reduced fidelity of the amplification and sequencing system of the DNA used [9-10].

In this context, it should be noted how, in an experiment in which a plasmid containing the variable portion of an immunoglobulin chain with a known sequence was sequenced using this method, a nucleotide misincorporation (C→T) was detected in a colony on the six colonies analyzed, for a total of 2,180 base pairs analyzed (corresponding to an error rate of 4.6×10⁻⁴ base pairs analyzed and equal to 16.6% of the clones analyzed) [1].

The ClonoSeq technique for identifying portions of clonal immunoglobulin sequences in commercially available biological samples is based on the combination of a multiplex PCR—which uses multiple primers aimed at amplifying all possible gene fragments of interest—and on the sequencing of short DNA fragments in order to identify the most abundant portions of nucleotide sequences within the variable region of the heavy and light chains of immunoglobulins. In particular, this method analyzes genomic DNA, not distinguishing between abortive gene rearrangements, which do not lead to the production of immunoglobulins, and productive rearrangements, which encode the immunoglobulins produced by the tumour clone. Furthermore, this method does not allow the whole variable sequence of clonal immunoglobulins to be obtianed. The applicability of this approach ranges from 79% to 91% of patients with multiple myeloma, as in a subset of cases the methods employed do not identify a sufficiently abundant or sufficiently unique sequence to qualify for tumour monitoring [11-14]. In a feasibility study of 36 patients with AL amyloidosis and no clinical evidence of concomitant multiple myeloma, all with measurable clonal disease based on serum electrophoresis and immunofixation and/or quantification of free light chains in the serum (with a median plasma cell infiltrate of the bone marrow of around 15%), the ClonoSeq method identified at least one traceable sequence in 31 of 36 patients (88.5%) [15].

The authors of the present invention have now developed a method for identifying the whole sequence of the variable region of the heavy and/or light chains of the different immunoglobulin isotypes expressed in a biological sample that combines the use of two-step reverse PCR with high-fidelity DNA polymerase, which enables an accurate amplification of the cDNA molecules of interest present in biological samples with real-time sequencing of single DNA molecules.

The method also allows part of the constant region of immunoglobulins to be identified.

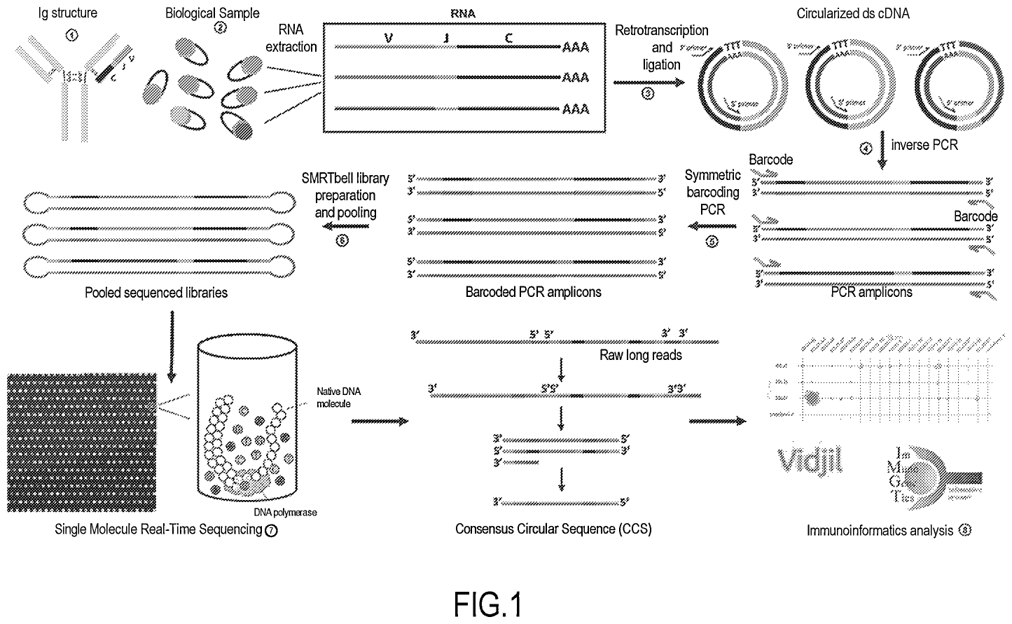

The method object of this patent application (called SMART M-Seq, single molecule real-time sequencing of the M protein—FIG. 1) also allows the sequences obtained on the basis of their relative abundance in the biological sample under examination, to be classified, allowing the ‘parallel analysis of a large number of biological samples. When applied to biological samples from patients suffering from a monoclonal gammapathy, SMART M-Seq allows the identification of monoclonal heavy and/or light chains (i.e. tumours).

The present invention therefore relates to a method for identifying the whole sequence of the variable region of the heavy and/or light chain of one or more immunoglobulin isotypes in a biological sample comprising the following steps:
i) extraction of intact RNA from said biological sample;
ii) reverse transcription of the RNA obtained in step i) and circularization of the ds cDNA thus obtained;
iii) two-step reverse PCR with high-fidelity DNA polymerase for accurate amplification with primer pairs directed against the constant region of circularized ds cDNA transcribed by the genes of said one or more immunoglobulin light and/or heavy chain isotypes;
iv) real-time sequencing of single DNA molecules, which allows the complete sequence of the variable region of one or more isotypes of the heavy and/or light chains of the immunoglobulins present in the biological sample, to be obtained.

The high-fidelity DNA polymerase used in step iii) is preferably selected from Q5 High-Fidelity 2× Master Mix, New England Biolabs, M0492S; Phusion Hot Start II High-Fidelity PCR Master Mix, ThermoFisher Scientific, F565L; Platinum Taq DNA Polymerase High-Fidelity, ThermoFisher Scientific, 11304011.

In a preferred embodiment of the method of the invention it is possible to have a further step v) for the classification of the isotypes identified in step iv) based on their relative quantity.

In a preferred embodiment of the invention the immunoglobulins are clonal, or cancerous.

The above-mentioned biological sample preferably comes from a patient's bone marrow. In an alternative embodiment the biological sample is a biopsy or it is preferably peripheral blood.

The method is preferably applied to biological samples from patients with monoclonal gammapathy.

These monoclonal gammopathies can be chosen from the group consisting of multiple myeloma, Waldenström's macroglobulinemia, monoclonal gammopathies of clinical significance (MGCS) or undetermined significance (MGUS), or systemic light chain amyloidosis (AL).

The primer pairs of step iii) directed against the constant region of circularized double-stranded cDNA transcribed by the genes of said one or more immunoglobulin light and/or heavy chain isotypes are preferably selected from the group consisting of:

| | |
|---|---|
| IGκ-fwd | /5AmMC6/GCAGTCGAACATGTAGCTGACTCAGGT |
| universal | CACTGCTCATCAGATGGCGGGAA (SEQ ID NO: 1) |
| IGκ-rev | /5AmMC6/TGGATCACTTGTGCAAGCATCACATCG |
| universal | TAGAAGAGCTTCAACAGGGGAGA (SEQ ID NO: 2) |
| IGλ-fwd | /5AmMC6/GCAGTCGAACATGTAGCTGACTCAGGT |
| universal | CACAGTGTGGCCTTGTTGGCTTG (SEQ ID NO: 3) |
| IGλ-rev | /5AmMC6/TGGATCACTTGTGCAAGCATCACATCG |
| universal | TAGGTCACGCATGAAGGGAGCAC (SEQ ID NO: 4) |
| IGγ-fwd | /5AmMC6/GCAGTCGAACATGTAGCTGACTCAGGT |
| universal | CACACCGGTTCGGGGAAGTAGTC (SEQ ID NO: 5) |
| IGγ-rev | /5AmMC6/TGGATCACTTGTGCAAGCATCACATCG |
| universal | TAGCTTCTCATGCTCCGTGATGC (SEQ ID NO: 6) |
| IGα-fwd | /5AmMC6/GCAGTCGAACATGTAGCTGACTCAGGT |
| universal | CACGGCTCCTGGGGGAAGAAGCCC (SEQ ID NO: 7) |
| IGα-rev | /5AmMC6/TGGATCACTTGTGCAAGCATCACATCG |
| universal | TAGCCTTCTCCTGCATGGTGGGCC (SEQ ID NO: 8) |

According to a preferred embodiment of the method of the invention, further comprising a verification step vi) in which the list of immunoglobulin heavy and/or light chains obtained from the analysis according to steps i)-iv) or i)-v) is used for the mapping of proteolytic peptides from serum and/or urinary proteins in a urine sample.

This further verification step can include a mass spectrometry analysis starting from the serum and/or urine sample that reveals the variant of the immunoglobulin chain most represented in the serum and/or urine sample under examination among those identified by SMART M-Seq in the starting sample (peripheral blood or marrow), allowing the identification or verification of the heavy and/or light monoclonal immunoglobulin chain even in those cases in which the clone is present in modest quantities in the starting sample, for technical reasons (e.g. bone marrow hemodilution) or biological reasons (e.g. peripheral blood sample from patient with MGCS).

The present invention will now be described for illustrative, but non-limiting, purposes, according to a preferred embodiment with particular reference to the attached figures, in which:

FIG. 1 shows a schematic representation of SMART M-Seq. The aim of the method is to obtain the whole sequence of the variable region of heavy and/or light immunoglobulin light chains (Ig) expressed in a given sample (\textcircled{1}) and to classify the sequences thus obtained on the basis of their relative abundance, in order to identify potential dominant (clonal) sequences, if, in the biological sample, there is a clone of B cells or plasma cells (\textcircled{2}). Total RNA is extracted from the biological sample, the mRNA is reverse-transcribed using an anchored oligo-d (T), and complementary double-stranded DNA (ds cDNA) is synthesized and circulated (\textcircled{3}). Two primers (in black) which appear at the constant region of the isotype of interest and which contain an adaptive sequence are used in the context of a reverse PCR using a high-fidelity DNA polymerase to obtain an amplicon comprising the whole variable region (\textcircled{4}). Two primers that match the adapter sequence and contain molecular barcodes identifying each sample are used for generating barcoded amplicons (\textcircled{5}) which are used for library preparation with “bell” adapters (\textcircled{6}) and subjected to sequencing of single DNA molecules in real time (\textcircled{7}). Bioinformatic approaches are used for analyzing the reads obtained and for extracting the so-called circular consensus sequences (CCS). The bioinformatic and immunogenetic analyzes, including Vidjil and IMGT/HighV-QUEST, are used for examining repertoires and identifying dominant clones (\textcircled{8}).

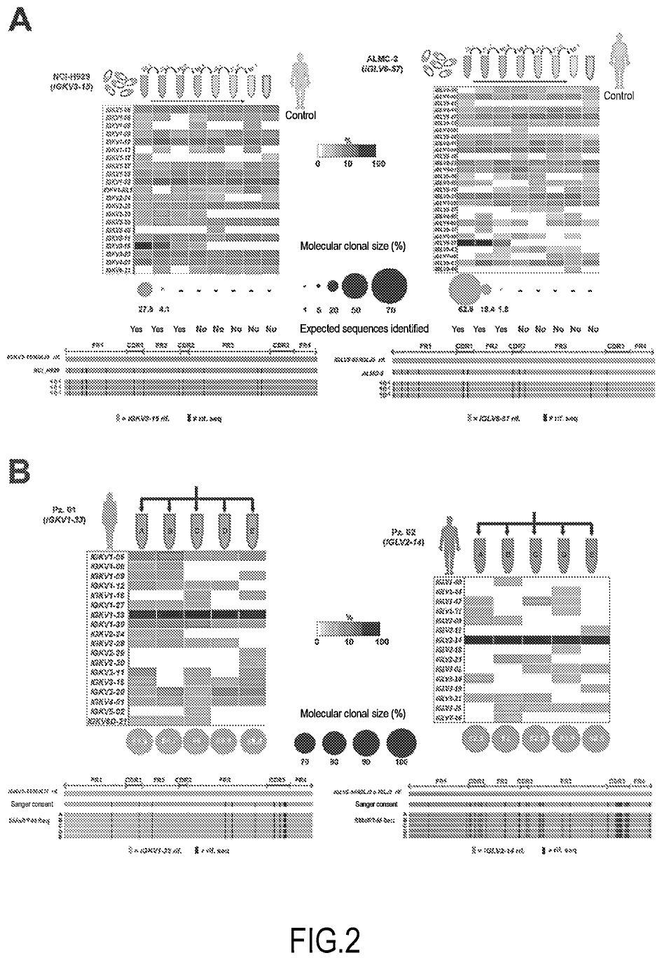

FIG. 2 shows the results of the validation of the SMART M-Seq method applied to immunoglobulin light chains of different isotypes. Panel A indicates the expression levels (in shades of grey) of different genes IGKV (left) and IGLV (right) evaluated by SMART M-Seq, starting from serial dilutions of total RNA from NCI-H929 cells, secreting the chain IGKV3-15 (on the left) or ALMC-2 cells, secreting IGLV6-57 (on the right,) in total RNA from bone-marrow mononuclear cells from a control subject with no evidence of lymphocyte/plasma cell clones. Panel B indicates the expression levels of different genes IGKV (left) and IGLV (right) evaluated by SMART M-Seq, starting from five replicated bone-marrow samples (A to E) from two patients (Pz. 01 and 02) suffering from AL amyloidosis, with a plasma cell clone secreting an IGKV1-33 (left) or IGLV2-14 (right) clonal light chain, respectively. In both panels A and B the scaled pie charts indicate the molecular size of the dominant clone identified in each sample tested. The minus sign (−) indicates samples in which no dominant clone has been identified with Vidjil. Below, sequence alignments of the clonotypic variable region of the light chain secreted by NCI-H929 and ALMC-2 cells (A) or the clonal light chain of patient 01 and patient 02 (B), evaluated by cloning and Sanger sequencing or by SMART M-Seq (indicated by the corresponding dilution, A, or by the 5 technical replicates, B), with the corresponding reference gene (ref.). The single mutations in the clonotypic light chain with respect to the corresponding germline gene are shown in black (≠ref. Seq.). FR: framework region, CDR: complementarity determining region, Pz.: patient.

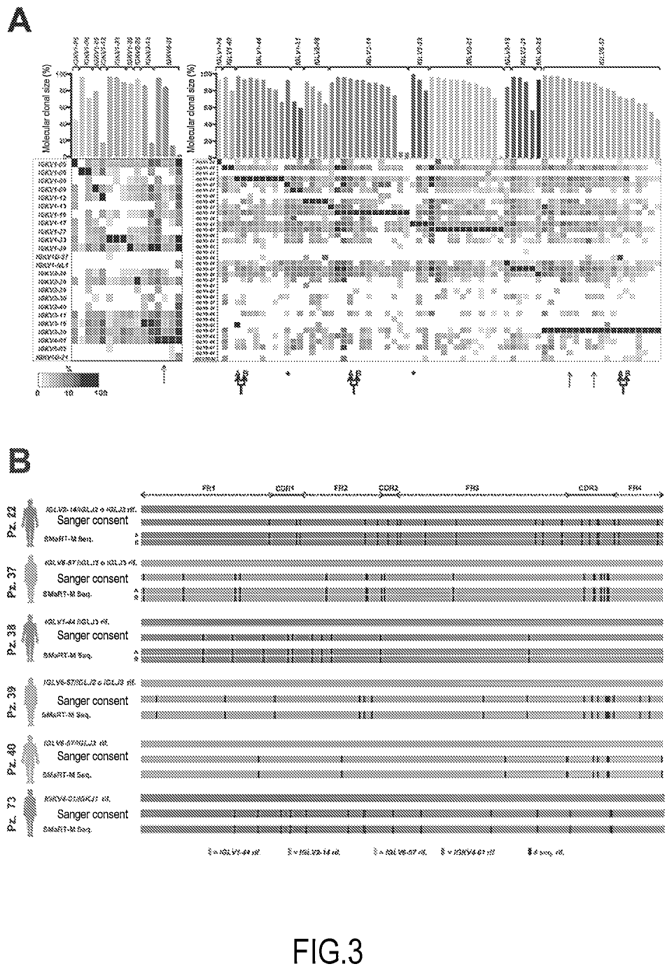

FIG. 3 shows the results relating to the application of the method according to the invention for the identification of the whole sequence of the variable region of the clonal light chain in a cohort of patients suffering from AL amyloidosis. Panel A reports the expression levels of different IGKV (left) and IGLV (right) genes evaluated by SMART M-Seq, starting from the diagnostic leftover of bone marrow of 84 patients with AL amyloidosis analyzed in parallel in a single cycle of sequencing. The bar graphs indicate the molecular size of the dominant clone identified by Vidjil's analysis in each sample tested. In two patients (*) the dominant clone was identified by IMGT/HighV-QUEST analysis. Three patients were analyzed in duplicate (arrows). Panel B illustrates the clonal light chain sequence alignments of six patients, assessed by Sanger cloning and sequencing or SMART M-Seq (A-B indicate technical duplicates) with the corresponding germline gene (ref.). The single mutations in the clonotypic light chain with respect to the corresponding germline gene are shown in black (ref. seq.). FR: framework region, CDR: complementarity determining region, Pz.: patient.

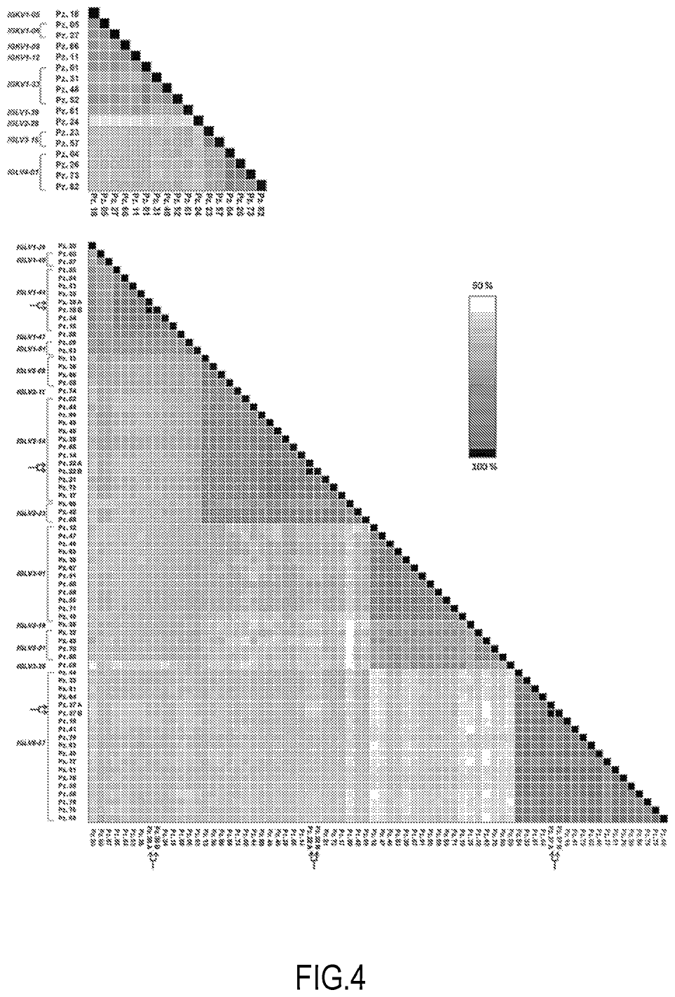

FIG. 4 shows the sequence homology of the clonal light chains in the cohort of AL patients analyzed. The Heatmap (in shades of grey) is shown representing the sequence homology levels of 86 patients with AL amyloidosis (17κ and 69λ,including patients 01 and 02). Three patients were analyzed in duplicate (arrows). Pz: patient.

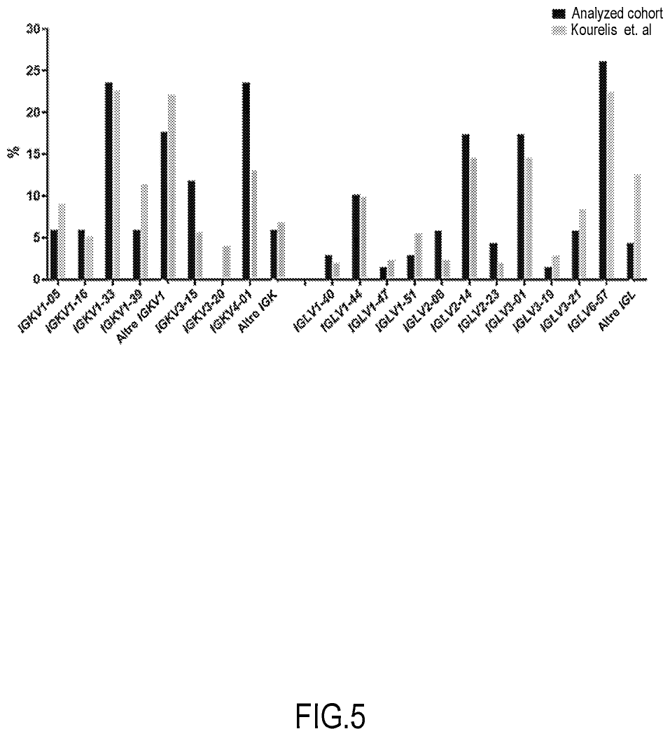

FIG. 5 shows the use of germline immunoglobulin genes in AL amyloidosis. A comparison is shown of the relative frequencies of the IGKV and IGLV genes in two cohorts of patients with systemic AL amyloidosis evaluated with SMART M-Seq (n=86, including patients 01 and 02) (in black) or by mass spectrometry (n=626) (in grey).

In order to better illustrate the invention, the following illustrative but non-limiting examples of the invention are provided.

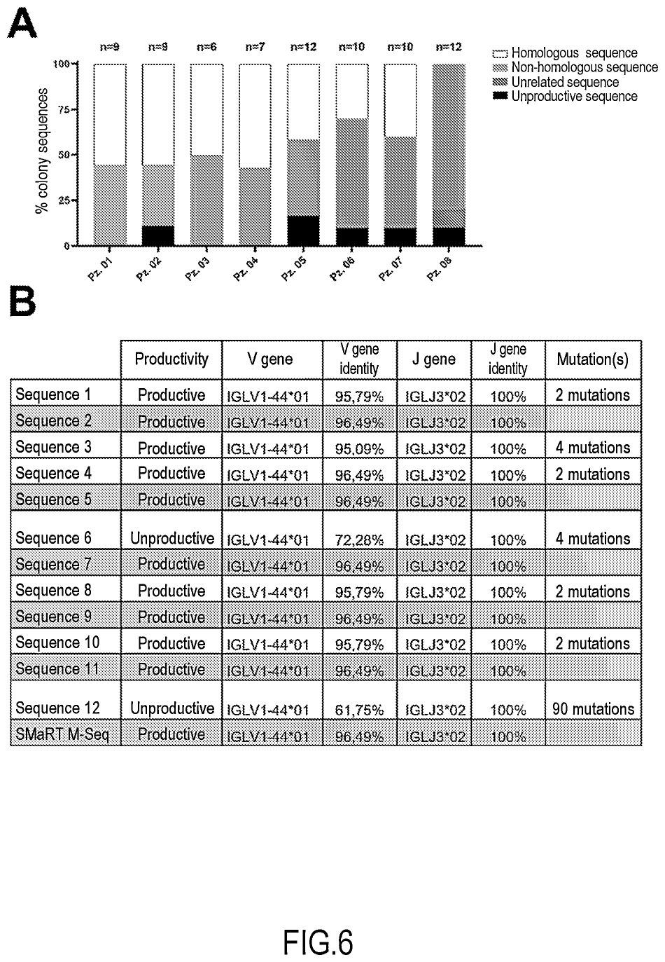

FIG. 6 shows a comparison of immunoglobulin sequencing results obtained with the classical single-step reverse PCR method with Taq polymerase, followed by cloning, bacterial transformation and Sanger sequencing of multiple bacterial colonies and the results obtained with SMART M-Seq, starting from bone-marrow blood samples from eight patients with AL amyloidosis.

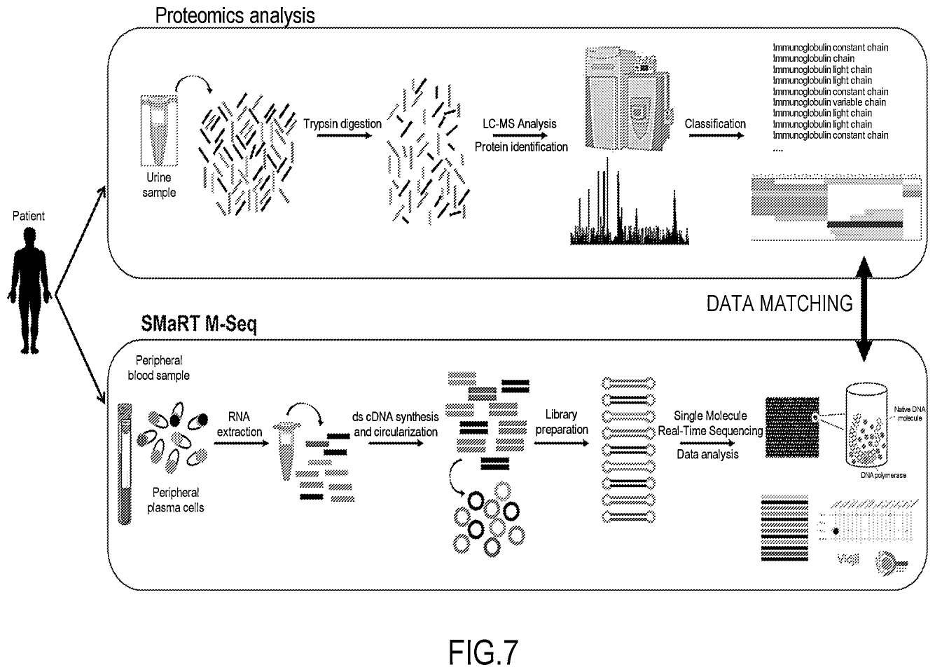

FIG. 7 shows a particular application of the SMART M-Seq method for the identification of the whole variable sequence of the clonal heavy and/or light immunoglobulin chain starting from a peripheral blood sample and a urine or serum sample of the patient with monoclonal gammapathy under consideration.

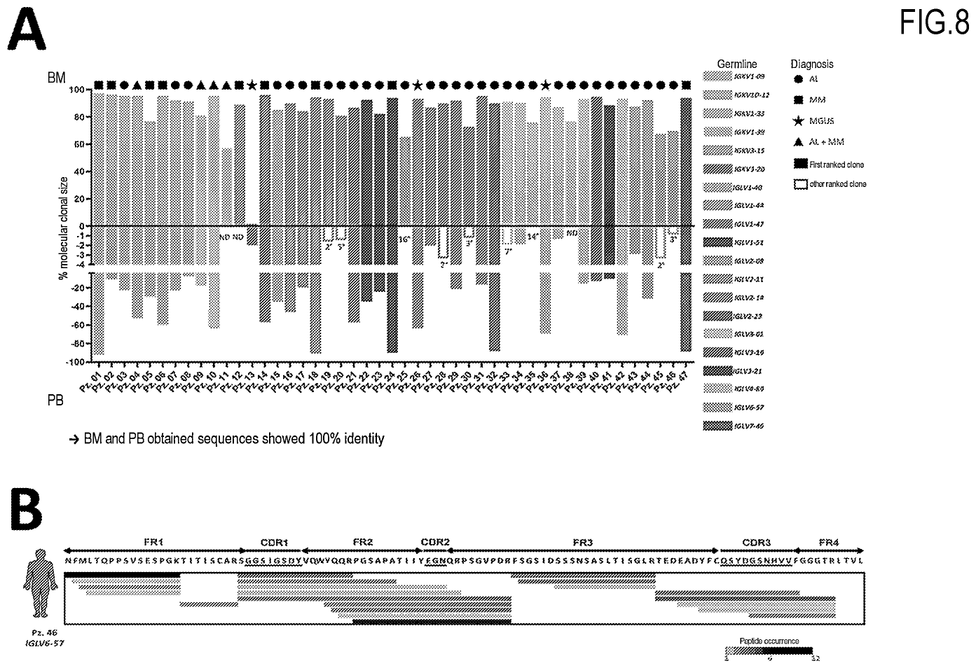

FIG. 8 shows the results obtained by using SMART M-Seq on peripheral blood for identifying the clonal light immunoglobulin chain in patients with monoclonal gammapathy, associated with proteomic analysis on urine.

The following illustrative but non-limiting examples of the invention are provided for a better illustration of the invention.

### EXAMPLE 1: Detailed Description of the SMART M-Seq Method

### Step 1: Extraction of RNA from the Biological Sample

Total RNA is extracted from the starting biological sample using TRIzol (Life Technologies, 15596026).

Other methods of RNA extraction from biological samples could also be employed, as long as the method used allows for the extraction of intact RNA molecules, as required by the subsequent reverse PCR step.

The biological sample was lysed with TRIzol, following the manufacturer's specific instructions. If the starting material is a cell suspension, the cell pellet is resuspended with TRIzol in relation to the quantity of starting material.

Incubation takes place for 5 minutes at room temperature to allow complete dissociation of the nucleoprotein complex. If necessary, the lysed sample can be stored at −80° C.

200 μL of chloroform are added for each ml of TRIzol used, and after vigorous stirring for 15 seconds the sample is incubated for 2-3 minutes at room temperature. This is followed by centrifugation of the sample at 4° C. for 15 minutes at 12,000 rcf. The aqueous phase is transferred to a new test-tube, care being taken not to collect the underlying phases which must be eliminated. 500 μl of isopropanol are added, stirred and then left on ice for 10 minutes. Also in this case, if the next step is not to be effected, the sample can be stored at −20° C. until the next day.

This is followed by centrifugation for 10 minutes at 4°° C. at 12,000 rcf. The total RNA precipitate is visible as an opaque white pellet at the bottom of the test-tube; the test-tube is kept on ice and the supernatant is removed. 1 mL of cold ethanol at 75% is added to the pellet. This is followed by a new centrifugation at 7,500 rcf for 5 minutes at 4° C. The supernatant is completely removed by aspiration with a micropipette. The residues of ethanol could cause a possible degradation of RNA therefore it is advisable to let the ethanol evaporate for 10 minutes or alternatively to centrifuge the sample at 7,500 rcf for 2 minutes at 4° C., aspirating the excess supernatant. 20-50 μL of water are added to the pellet and gently resuspended. The intact RNA sample extracted is kept on ice.

At this point, the quantity of RNA extracted is determined by means of a spectrophotometer and/or fluorometer.

Before proceeding with the next synthesis step of double-stranded cDNA (ds cDNA) it is advisable to evaluate the integrity of the RNA extracted by an electrophoretic run on agarose gel or capillary electrophoresis.

### Step 2: Synthesis, Purification and Precipitation of ds cDNA

The following reagents were used:
Anchored OligodT (0.5 μg/μL): Invitrogen 12577011
dNTP mix 10 mM (dATP, dCTP, dGTP, dTTP): Invitrogen 10297018
DTT 0.1 M: Invitrogen Y00147
First Strand Buffer 5×: Invitrogen P/N Y02321
RNaseOUT (40 U/μL): Invitrogen 10777019
Superscript II Reverse Transcriptase (200 U/μL): Invitrogen 18064014
Second Strand Buffer 5×: Invitrogen 10812014
RNasi H (2 U/μL): Invitrogen 18021071
E. Coli DNA Polimerase I (10 U/μL): Invitrogen 18010017
E. Coli DNA ligase (10 U/μL): Invitrogen 18052019
T4 DNA polimerase (5 U/μL): Invitrogen 18005017

The total RNA extracted in the previous step is reverse-transcribed into a double-stranded cDNA. Reverse transcription takes place using an anchored oligod (T). 500-1,000 ng of RNA were used and brought to a total volume of 10 μL with water by adding the following reagents in order:
1 μL of anchored oligodT (0.5 μg/μL)
1 μL of RNase out enzyme (40 U/μL)

This is followed by incubation at 70° C. for 10 minutes. At the end of the incubation, a short centrifugation is applied and the test-tube is placed on ice. The following reagents are added in order:
4 μL of First strand reaction buffer 5×
2 μL of DTT 0.1 M
1 μL of dNTP mix 10 mM

Before incubation, the test-tube is shaken gently and a short centrifugation is applied in order to mix the reagents.

This is followed by incubation at 45° C. for 2 minutes in order to equilibrate the temperature.

1 μL of Superscript II Reverse Transcriptase (200 U/μL) is added and incubated at 45° C. for 60 minutes. The total volume of the reaction for the synthesis of the first cDNA strand is equal to 20 μL.

At the end of the incubation, a short centrifugation is applied and the test-tube is placed on ice.

This is followed by the synthesis of the second cDNA strand by adding the following reagents in order:
93 μL of water.
30 μL of Second strand reaction buffer 5×
3 μL of dNTP mix 10 mM
1 μL of E. Coli DNA ligase (10 U/μL)
2 μL of E. Coli DNA Polimerase I (10 U/μL)
1 μL of E. Coli DNA RNasi H (2 U/μL)

Before incubating, the test-tube is shaken gently and a short centrifugation is applied to mix the reagents.

The total volume of the reaction for the synthesis of the second cDNA strand is equal to 150 μL. This is followed by incubation at 16° C. for 2 hours.

At the end of the incubation, 1 μL of T4 DNA Polymerase (5 U/μL) is added and incubated at 16°° C. for a further 5 minutes. A short centrifugation is applied and the test-tube is placed on ice.

160 μL of a mixture of phenol-chloroform-isoamyl alcohol (25:24:1) are added and shaken vigorously for 15 seconds. The mixture is left to incubate for 2-3 minutes until phase separation.

The sample is centrifuged at 4° C. for 5 minutes at 12,000 rcf. Only the upper phase is transferred to a new test-tube, care being taken not to withdraw the underlying phases.

160 μL of STE buffer are added to the initial test-tube, shaken vigorously for 15 seconds and incubated for 2-3 minutes until phase separation. The sample is centrifuged at 4° C. for 5 minutes at 12,000 rcf.

Only the upper phase is transferred to the new test-tube, care being taken not to withdraw the underlying phases which will be eliminated. The total volume of the new test-tube should be approximately 300 μL.

At this point the ds cDNA is precipitated by adding the following reagents in order:
100 μL of ammonium acetate
750 μL of cold ethanol

The mixture is stirred vigorously and centrifuged at 4° C. for 20 minutes at 12,000 rcf. The supernatant is gently removed and the pellet is resuspended with 500 μL of cold ethanol at 75%. This is centrifuged at 4° C. for 10 minutes at 12,000 rcf and the whole supernatant is gently removed. Also in this case, the ethanol residues could cause a possible degradation of the ds cDNA, so it is advisable to allow the ethanol to evaporate for 10 minutes or alternatively centrifuge the sample at 12,000 rcf for 2 minutes at 4° C. aspirating the excess supernatant.

The pellet is resuspended in 10 μL of water by gently shaking the test-tube and applying a short centrifugation. If the next step is not to be effected, the sample can be stored at −20° C.

### Step 3: Circularization of the ds cDNA

Double-stranded cDNA is circulated using a DNA ligase (T4 DNA ligase (1 U/μL) Invitrogen 15224017).

The reaction is prepared by adding the following reagents to a new test-tube:
4 μL of water
2 μL of T4 DNA ligase 5× buffer
3 μL of ds cDNA
1 μL of T4 DNA ligase (1 U/μL)

Before proceeding with the incubation, the test-tube is shaken gently and a short centrifugation is applied in order to homogenize the reagents. It is incubated at 14° C. for 16-20 hours.

### Step 4: Amplification of the Target Region of the Immunoglobulin Chain of Interest by Reverse PCR

The target region of the immunoglobulin chain of interest is amplified from the ds cDNA by two-step reverse PCR using a high-fidelity DNA polymerase. In the first PCR step, the immunoglobulin isotype of interest is amplified and, at the same time, universal adapters are incorporated, to allow subsequent labelling of the amplicons with a special molecular barcode, according to the Pacific Biosciences sequencing protocol. In the second PCR step, a second amplification is performed and, at the same time, the molecular barcode is incorporated, according to the Pacific Biosciences sequencing protocol.

The following reagents are used:
Q5 High-Fidelity 2X Master Mix (New England Biolabs, M0492S)
universal target-specific forward and reverse primers (listed in Table 1 below)
Barcoded Universal F/R Primers Plate-96 wells (Pacific Biosciences, 101-629-100)
Water (DNase and RNase free)
MinElute Gel Extraction Kit (Qiagen, 28604)

TABLE 1

| Primer | Primer Sequence (5′ -> 3′) |
|---|---|
| IGκ-fwd | /5AmMC6/GCAGTCGAACATGTAGCTGACTCAGGTCAC |
| universal | TGCTCATCAGATGGCGGGAA (SEQ ID NO: 1) |
| IGκ-rev | /5AmMC6/TGGATCACTTGTGCAAGCATCACATCGTAG |
| universal | AAGAGCTTCAACAGGGGAGA (SEQ ID NO: 2) |
| IGλ-fwd | /5AmMC6/GCAGTCGAACATGTAGCTGACTCAGGTCAC |
| universal | AGTGTGGCCTTGTTGGCTTG (SEQ ID NO: 3) |
| IGλ-rev | /5AmMC6/TGGATCACTTGTGCAAGCATCACATCGTAG |
| universal | GTCACGCATGAAGGGAGCAC (SEQ ID NO: 4) |
| IGγ-fwd | /5AmMC6/GCAGTCGAACATGTAGCTGACTCAGGTCAC |
| universal | ACCGGTTCGGGGAAGTAGTC (SEQ ID NO: 5) |
| IGγ-rev | /5AmMC6/TGGATCACTTGTGCAAGCATCACATCGTAG |
| universal | CTTCTCATGCTCCGTGATGC (SEQ ID NO: 6) |
| IGα-fwd | /5AmMC6/GCAGTCGAACATGTAGCTGACTCAGGTCAC |
| universal | GGCTCCTGGGGGAAGAAGCCC (SEQ ID NO: 7) |
| IGα-rev | /5AmMC6/TGGATCACTTGTGCAAGCATCACATCGTAG |
| universal | CCTTCTCCTGCATGGTGGGCC (SEQ ID NO: 8) |

The first PCR reaction (PCR 1) is prepared for each sample by adding the following reagents in a final volume of 25 μL:
12.5 μL of Q5 High-Fidelity 2× Master Mix
1.25 μL of Universal primer forward target-specific (10 μM)
1.25 μL of Universal primer reverse target-specific (10 μM)
8 μL of water
2 μL of circularized ds cDNA previously obtained

The test-tube is shaken gently and a short centrifugation is applied to homogenize the reagents.

A duplicate is created for each sample in order to minimize the impact of any nucleotide base incorporation errors during the first PCR cycles.

Amplification is carried out under the conditions shown hereunder:

TABLE 2

| Step | T° C. | Time | Cycles |
|---|---|---|---|
| Initial denaturation | 98° C. | 30 seconds | |
| Denaturation | 98° C. | 10 seconds | 15 |
| Primer pairing | 72° C. | 30 seconds | |
| Extension | 72° C. | 30 seconds | |
| Final extension | 72° C. | 60 seconds | |

At the end of the amplification, the duplicates of each sample are combined. The amplicon, however, can be stored at −20° C. for a few days.

The second PCR reaction (PCR 2) is then prepared for each sample by adding the following reagents in a total volume of 25 μL:
12.5 μL of Q5 High-Fidelity 2× Master Mix
2.5 μL of F/R barcode primer
8 μL of water
2 μl of template from PCR 1

The test-tube is shaken gently and a short centrifugation is applied to mix the reagents.

The second amplification is carried out under the conditions illustrated in the following Table:

TABLE 3

| Step | T° C. | Time | Cycles |
|---|---|---|---|
| Initial denaturation | 98° C. | 30 seconds | |
| Denaturation | 98° C. | 10 seconds | 15 |
| Primer pairing | 72° C. | 30 seconds | |
| Extension | 72° C. | 30 seconds | |
| Final extension | 72° C. | 60 seconds | |

At the end of the amplification, the PCR products are displayed on an agarose gel. The bands from the gel are excised and the amplicons are purified by Mini Elute Gel extraction kit (Qiagen) in accordance with the guidelines of the kit.

The quantification of the individual amplicons is carried out by means of a fluorometer (Qubit).

If two or more samples are to be sequenced in parallel, a pooling of the amplicons of the different samples in question is effected, combining equal quantities of amplicon for each sample, following the guidelines of Pacific Biosciences.

### Step 5: Sequencing of Single DNA Molecules in Real-Time

The amplicon or pooling of amplicons generated in the previous steps will be used for the creation of the sequencing library using the SMRT bell adapters, subjected to real-time sequencing of single DNA molecules for the generation of the CCS circular consensus sequences), in accordance with the guidelines of Pacific Biosciences.

The SMRT bell library is prepared in accordance with the manufacturer's guidelines (Pacific Biosciences).

The bioinformatics analysis for the generation of circular consensus sequences (CCS) and demultiplexing are also carried out according to the guidelines of Pacific Biosciences.

### Step 6: Immunoglobulin Analysis by Bioinformatics/Immunogenetic Analyses

For each sequenced sample, the relative file containing the CCS sequences in FASTA format is subjected to bioinformatics/immunogenetic analyses.

The sequences of each sample are loaded in FASTA format using the Vidjil software (http://www.vidjil.org/).

For each sample, the analysis is started by selecting the “V(D)J recombinations” section and choosing the “multi+inc+xxx” analysis (default: multi-locus, with same incomplete/unusual/unexpected recombinations).

At the end of the analysis, the result is displayed for each sample by selecting the isotype of the sequenced chain; In this way, the list of all clones identified is obtained, with their relative molecular clonal dimensions.

The clones obtained are sorted according to the relative frequency (“Sort by size” field). In the case of a sample obtained from a patient with a monoclonal gammapathy, the sequence originating from the clone is typically the first sequence obtained in terms of relative frequency, with a molecular clonal size greater than 1% and greater than twice the second more frequent sequence identified. A more complex clonal pattern may be found in patients with biclonal gammapathy, in patients undergoing bone-marrow engraftment after haematopoietic stem cell transplantation or in other clinical situations.

At the end, the clonal sequence obtained in terms of productivity of the immunoglobulin chain sequenced through the IMGT/V-QUEST portal (http://www.imgt.org) is verified by selecting the species and the type of isotype sequenced, loading the clonal sequence in FASTA format.

The analysis of the Vidjil software can be repeated on IMGT/HighV-QUEST, especially if the analysis of the sequences of a sample results in an alert signal (yellow-orange triangle with exclamation point, “Few sequences analyzed”, or red triangle with exclamation point “Very few sequences analyzed” the result of few sequences analyzed) or the first clone found for molecular clonal size is indicated as “smaller clones”. Through the IMGT/HighV-QUEST portal, it is possible to upload the sequences obtained in FASTA format, launch the analysis by selecting the species and the sequenced isotype.

### EXAMPLE 2: Validation of the SMART M-Seq Method

The validation of the SMART M-Seq method described in the previous example was effected, studying its accuracy, repeatability and sensitivity.

For this purpose, the human myeloma plasma cell line NCI-H929 was used, and the human amyloidogenic plasma cell line ALMC-2 [17], which secrete an immunoglobulic light chain κ or λ, respectively.

Using standard molecular biology techniques, the sequence of the whole variable region of the κ light chain expressed by the cell line NCI-H929 was determined, which was found to originate from an IGKV3-15 gene (data not shown).

Conversely, for the ALMC-2 cell line, the sequence of the whole variable region of the light chain λ expressed was included in the original description of this

cell line [17]. The λ light chain sequence expressed of the ALMC-2 cells in use was experimentally verified, confirming the origin from an IGLV6-57 gene and 100% identity with the published sequence (data not shown).

In order to test the sensitivity and accuracy of SMART M-Seq in detecting the whole sequence of the variable region of clonal immunoglobulins, 1 volume of total RNA from the human plasma cell line NCI-H929 or ALMC-2 was combined with 9 volumes of total RNA from the bone marrow of a subject with no detectable plasma cell clones.

6 further serial dilutions 1 to 10 were then prepared, again in total RNA from the bone marrow of the control subject (thus obtaining a final dilution of the RNA of the plasma cell line from 10⁻¹ to 10⁻⁷) in order to mimic samples of bone marrow containing a plasma cell clone expressing a progressively smaller κ or λ light chain. This procedure produced 16 samples (8 samples for κ light chain sequencing and 8 samples for λ light chain sequencing, including 10⁻¹ to 10⁻⁷ dilutions and healthy donor, for each type of light chain).

The 16 RNA samples thus obtained were subjected to amplification, addition of molecular barcodes, pooling (together with 10 additional samples, as specified below) and real-time sequencing of single DNA molecules on the Pacific Biosciences RSII platform. After demultiplexing, a median of 915 sequences per sample was obtained (interquartile range: 757-1,204 sequences). Each sample was analyzed separately with Vidjil [18] to blindly identify the dominant clonal sequences, i.e. without exploiting an a priori knowledge of the clonal sequence of the plasma cell line used for the generation of the sequenced samples. In parallel, the individual FASTA files containing all the sequences identified in a given sample were inspected individually to verify the presence and relative frequency of the clonal sequence predicted based on an a priori knowledge of the sequence itself.

Without an a priori knowledge of the expected clonal sequence, Vidjil clonal analysis was able to identify a dominant clonal sequence up to 10⁻² dilution for bone-marrow samples spiked with NCI-H929 plasma cell line RNA and subjected to κ light chain sequencing and up to 10⁻³ dilution for bone-marrow samples spiked with ALMC-2 cell RNA and subjected to λ light chain sequencing (FIG. 2). It should be noted that the dominant clonal sequence identified by Vidjil analysis in the five samples that gave positive results (10⁻¹ and 10⁻² for the κ light chain sequence and 10⁻¹ to 10⁻³ for the light chain sequence λ), showed in all cases a 100% identity with respect to the nucleotide sequence predicted based on the sequence of the κ and λ light chains expressed by the NCI-H929 line and the ALMC-2 line respectively, demonstrating 100% accuracy in the determination of the clonal sequence at the level of the single base pair analyzed. Furthermore, the molecular clonal size obtained, which is a measurement of the relative frequency of clonal sequences in a given sample, was progressively lower in samples with a decreasing concentration of the original RNA of the plasma cell line, as expected (FIG. 2).

In parallel, in order to test the reproducibility of SMART M-Seq for the detection of clonal immunoglobulin genes in real bone-marrow samples from patients with monoclonal gammapathy, two patients were selected with AL amyloidosis upon diagnosis (patient 01, with a plasma cell with restriction of κ light chains and a plasma cell infiltrate in the bone marrow equal to 6%, and patient 02, with a plasma cell clone with restriction of λ light chains and a plasma cell infiltrate in the bone marrow equal to 11%). The nucleotide sequence of the variable region of their clonal light chain κ or λ, respectively, was obtained through a standard approach consisting of amplification by reverse PCR, TOPO-TA cloning, E. coli transformation and sequencing of multiple colonies [1]. These analyzes demonstrated that patient 01 expresses a clonal κ light chain deriving from the germline gene IGKV1-33, whereas patient 02 expresses a clonal light chain λ deriving from the germline IGLV2-14 gene. These 2 bone-marrow RNA samples from patients 01 and 02 were subsequently divided into 5 different test-tubes for each patient, and the resulting 10 samples (5 replicate RNA samples for patient 01 and 5 replicate RNA samples for patient 02) were then processed independently according to the SMART M-Seq protocol. In particular, the pooling and sequencing for these 10 samples took place simultaneously with the 16 samples from the serial dilution experiment mentioned above. After demultiplexing, a median of 730 sequences per sample was obtained for these 10 samples (interquartile range: 603-953 sequences). Again, each sample was analyzed separately with Vidjil to blindly identify dominant clonal sequences, without exploiting an a priori knowledge of the patient-specific sequence determined by conventional methods. It should be noted that for both patients analyzed, the clonal sequence obtained by SMaRT M-Seq was identical in the 5 samples examined, with a 100% identity with the predicted κ or λ clonal sequence, previously obtained for each patient by conventional methods (FIG. 2), further confirming the 100% accuracy in the determination of the clonal sequence at the level of the single base pair analyzed.

Furthermore, Vidjil was used for determining the molecular clonal size of each sample, as a result of the relative frequency of the clonal sequence with respect to all sequences obtained in each sample. The five replicated samples from patient 01 resulted in a sequence-based molecular clonal size of 89% (variation coefficient, CV: 0.5%), whereas the five replicated samples from patient 02 resulted in a molecular clonal size of 92.9% (CV: 0.7%) (FIG. 2). Collectively, these results demonstrate that SMaRT M-Seq can accurately and reproducibly identify the whole variable region sequence of clonal immunoglobulin genes from a biological sample. In all cases, in fact, SMART M-Seq allowed clonal sequences to be identified with 100% identity with sequences obtained with conventional methods. Furthermore, the method showed a variation coefficient of <1% in determining the molecular clonal size of the dominant clone and showed a sensitivity governed by the number of total sequences per sample obtained during sequencing (within the range of 10⁻²-10⁻³ in the present experiment) and therefore capable of being increased by analyzing a smaller number of samples in parallel and/or by using a platform with a greater sequencing depth.

SMART M-Seq was subsequently used for the identification of clonal immunoglobulin sequences from bone-marrow mononuclear cells of a cohort of patients with systemic AL amyloidosis. To this end, 89 patients with systemic AL amyloidosis or suspected systemic AL amyloidosis were analyzed, with a residual bone-marrow blood sample after completion of diagnostic procedures available for research purposes.

SMART M-Seq was effected on the cohort of 89 patients, who were analyzed in parallel. In six randomly selected patients (patients 22, 37, 38, 39, 40 and 73), the amyloidogenic light chain sequence expressed was also obtained through a standard cloning and sequencing approach for comparison purposes [1]. In 3 of these patients (patients 22, 37, 38) SMART M-Seq was effected in duplicate RNA samples, processed and analyzed independently, whereas the remaining 86 patients in the cohort were analyzed as individual samples. On the whole, 92 samples underwent amplification, molecular barcode incorporation, pooling and were then analyzed in a single sequencing run using the Pacific Biosciences Sequel platform, following the SMART M-Seq protocol. After demultiplexing, a median of 3,118 sequences per sample was obtained (interquartile range: 2,554-3,671). Each sample was analyzed separately with Vidjil to identify the dominant clonal sequence and molecular clonal size.

In all 6 patients for whom the clonal light chain sequence was also obtained with conventional methods, SMART M-Seq correctly identified the clonal light chain sequence expected, with 100% identity compared to the sequence obtained with standard cloning and sequencing approaches (FIG. 3). Furthermore, for the 3 patients analyzed in duplicate (patients 22, 37 and 38), the molecular clonal sizes based on the sequence of the two technical replicates were highly comparable (FIG. 3). These results further confirm the accuracy and repeatability of SMART M-Seq.

Of the 89 patients sequenced, in 84 patients, a final diagnosis of systemic AL amyloidosis could be established, whereas the remaining 5 cases could not confirm the diagnosis of systemic AL amyloidosis due to lack of histological evidence of amyloid deposits or because an alternative diagnosis had been made.

In the 84 patients sequenced with a final diagnosis of AL amyloidosis, the amyloidogenic light chain was κ-type in 16 cases (19%) and λ-type in 68 cases (81%). The median plasma cell infiltration of the bone marrow was 9% (range 1-30%). In 5 of these patients (patients 12, 32, 35, 44 and 47), electrophoresis with immunofixation of serum and urine effected with standard methods gave a negative result and the κ/λ ratio of the concentration of serum free light chains was found to be normal, demonstrating the presence of a particularly small plasma cell clone, difficult to detect. In these 5 cases, the presence of a monoclonal gammapathy was demonstrated by electrophoresis with immunofixation on high-resolution agarose gel [19-20], possibly associated with multiparametric flow cytometry of marrow blood performed with the standard method or with the high-sensitivity next-generation method (Next Generation Flow, NGF, sensitivity of 10-6) [15].

In this experiment, SMART M-Seq allowed a clonal sequence of immunoglobulin light chains to be identified in all 84 patients (median molecular clonal size: 88.3%, interquartile range: 70.7-93%). The molecular clonal size identified by SMART M-Seq showed a significant correlation with the percentage of plasma cell infiltrate in the bone marrow (p<0.0001) (data not shown).

The clonal sequences obtained from 86 patients with confirmed AL amyloidosis (17κ and 69λ, also including patients 01 and 02) were aligned. Each patient's sequence turned out to be unique, as expected (FIG. 4).

Of those 5 of the 89 patients sequenced in which it was not possible to confirm a final diagnosis of AL amyloidosis, in 2 cases (patient 07 and 16) there was a monoclonal gammapathy, whereas in the remaining 3 cases (patients 03, 08 and 29) the tests performed (including electrophoresis with immunofixation of serum and urine and quantification of serum free light chains) excluded the presence of a medullary clone. In this context, SMART M-Seq identified a clonal sequence in both patients with a monoclonal gammapathy (with a clonal molecular size of 53.7% and 4.3%, respectively) and in none of the 3 patients with no detectable plasma cell clone. (data not shown).

After obtaining the whole variable sequence of immunoglobulin light chains produced by the plasma cell clone of each patient analyzed, the genes/germline alleles IGKV and IGLV used in each case were determined, using the IMGT/V-QUEST platform. Among the 86 patients with systemic AL amyloidosis sequenced by SMaRT M-Seq (including patients 01 and 02), the most common germline κ genes were the IGKV1-33 and IGKV4-01 genes (24% each of the 17κ AL patients) and the most common germline λ genes were IGLV6-57 (26% of 69λ AL patients), IGLV2-14 (17%), IGLV3-01 (17%) and IGLV1-44 (10%). On the whole, the most frequent germline λ and κ genes (IGLV6-57, IGLV2-14, IGLV3-01, IGLVI-44, IGKV1-33 and IGKV4-01) together accounted for 66% of all the clones.

The composition and relative frequencies of the κ and λ germline genes used in the whole cohort of 86 patients analyzed by SMART M-Seq were in agreement with the results of Kourelis et al. [21], who studied the use of the germline gene in a larger cohort of AL patients using liquid chromatography/tandem mass spectrometry (LC-MS) in biopsies of tissues with amyloid deposits, while not identifying the whole sequence of the variable region of amyloidogenic light chains [21] (FIG. 5). This observation further corroborates the capacity demonstrated of SMART M-Seq in correctly identifying the clonal immunoglobulin sequence in biological samples from patients with monoclonal gammapathy.

### EXAMPLE 3: Comparative Data with the Classical Method of Reverse PCR, Cloning and Sanger Sequencing

The combination of steps iii) and iv) in the method according to the invention confers accuracy and sensitivity to the method according to the invention.

In the methods described in the state of the art, sequencing takes place after cloning. Cloning followed by sequencing introduces errors as bacteria have an error-prone DNA replication apparatus.

Furthermore, there is a sensitivity issue in prior art methods, especially for detecting the clone in peripheral blood (liquid biopsy approach) or for analyzing diluted samples.

The sensitivity is dictated by how many sequences are analyzed. Finally, it is not possible to analyze the samples in parallel.

Comparative experiments were conducted that demonstrate the greater accuracy of the combination of steps iii) and iv) of the method according to the invention compared to prior methods that contemplate the sequencing and cloning steps.

In particular, the results of immunoglobulin sequencing obtained with the classical single-step reverse PCR method with Taq polymerase were compared, followed by cloning, bacterial transformation and Sanger sequencing of multiple bacterial colonies, according to the protocol reported in [1], and the results obtained with SMART M-Seq, starting from bone-marrow blood samples from eight patients with AL amyloidosis.

For the classical sequencing method, through cloning and Sanger sequencing, from 6 to 12 bacterial colonies per patient/bone-marrow sample were analyzed. The dominant clonal sequence was defined as the consensus sequence among the sequences obtained. FIG. 6, panel A shows a summary of the results obtained by the classical Sanger cloning and sequencing method. In five of the 8 patients analyzed, one or more sequences among those obtained were found to be non-productive, probably due to artifacts of PCR and/or sequencing. In seven of the eight patients analyzed, one or more sequences were identified that differed from the consensus sequence for one or more mutations, again probably due to PCR and/or sequencing artifacts. In one case (patient 8), none of the 12 sequences analyzed completely corresponded to the consensus sequence, each differing from the consensus sequence for one or more mutations. In all cases, the consensus sequence identified corresponded 100% to the sequence identified by SMaRT M-Seq. FIG. 6, panel B shows the details of the sequences obtained for a representative patient among those analyzed.

### EXAMPLE 4: Combination of the SMART M-Seq Method and Proteomic Analysis

FIG. 7 shows the particular application of the SMART M-Seq method for the identification of the whole variable sequence of the clonal heavy and/or light immunoglobulin chain starting from a peripheral blood sample and a urine or serum sample of the patient with monoclonal gammapathy under consideration.

The SMART M-Seq method was effected starting from mononuclear cells (or alternatively from buffy coat) obtained from peripheral blood, allowing a list of immunoglobulin sequences expressed in the biological sample under examination to be obtained.

In parallel, a proteomic analysis on urine (or serum) is carried out by enzymatic digestion of proteins (e.g. trypsin digestion) and analysis with liquid chromatography and mass spectrometry (LC-MS/MS). The list of immunoglobulin heavy and/or light chains obtained by SMART M-Seq is used for mapping the spectra obtained by mass spectrometry analysis.

The heavy and/or light chain most identified by mapping the peptides obtained with mass spectrometry represents the clonal heavy and/or light chain.

FIG. 8 shows the results obtained using SMART M-Seq on peripheral blood for identifying the clonal light immunoglobulin chain in patients with monoclonal gammapathy, associated with proteomic analysis on urine.

A cohort of 47 patients was analyzed (31 with AL amyloidosis, 9 with multiple myeloma, 4 with multiple myeloma and AL amyloidosis and 3 with MGUS). Bone-marrow blood was analyzed to uniquely identify the clonal immunoglobulin light chain.

FIG. 8, panel A shows a summary of the results obtained in the 47 patients analyzed. In 35 of the 47 patients analyzed, SMART M-Seq performed on peripheral blood identified the clonal light chain as the dominant light chain (most abundant clone based on the number of sequences obtained out of the total sequences obtained for each peripheral blood sample).

In 9 out of 47 cases the clonal light chain was present among the immunoglobulin light chains identified by SMART M-Seq in peripheral blood, but this sequence was not in first place for relative abundance, but in a variable position from position number 2 to position number 16, depending on the case. The clonal light chain was not present among the immunoglobulin light chains identified by SMART M-Seq in peripheral blood in only 3 patients out of the 47 patients analyzed.

FIG. 8, panel B shows the proteomic investigations on urine for the identification (or confirmation) of the clonal light chain. The list of immunoglobulin light chains obtained by SMART M-Seq was used for mapping the peptides obtained by mass spectrometry of urinary proteins after digestion with trypsin. The light chain most present in the urine sample under examination on the basis of the results of the mapping of the peptides was found to correspond to the clonal immunoglobulin light chain of the patient under examination, as previously detected by means of bone-marrow blood analysis using SMART M-Seq.

### BIBLIOGRAPHY

[1] Perfetti, V. et al. Anal Biochem 239, 107-109, 1996.
[2] Garofalo, M. et al. Nat Commun 12, 3532, 2021.
[3] Uematsu, Y. et al. Immunogenetics, 34, 174-178,1991.
[4] Inaba, T. et al. Int Immunol 3, 1053-1057, 1991.
[5] Menzel, U. et al. PLoS One 9, e96727, 2014.
[6] Fujii, S. et al. J Mol Biol 289, 835-850, 1999.
[7] Perfetti, V. et al. Blood 100, 948-953, 2002.
[8] Perfetti, V. et al. Blood 119, 144-150, 2012.
[9] Speksnijder, A.G. et al. Appl Environ Microbiol 67, 469-472, 2001
[10] Rico-Torres, C.P. et al. Infect Genet Evol 101, 105283, 2022
[11] Ching, T. et al. BMC Cancer 20, 612, 2020.
[12] Martinez-Lopez, J. et al. Blood 123, 3073-3079, 2014.
[13] Yao, Q. et al. Front Oncol 10, 611021, 2020.
[14] Hultcrantz, M. et al. Clin Cancer Res 28, 2160-2166, 2022
[15] Sarosiek, S. et al. Blood Cancer J. 11, 117, 2021.
[16] Gazdar, A. F. et al. Blood 67, 1542-1549, 1986.
[17] Arendt, B. K. et al. Blood 112, 1931-1941, 2008.
[18] Duez, M. et al. PLoS One 11, e0166126, 2016.
[19] Merlini, G. et al. Clinical chemistry andlaboratory medicine: CCLM/FESCC 39, 1025-1028, 2001.
[20] Palladini, G. et al. Clinical chemistry 55, 499-504,2009.
[21] Kourelis, T. V. et al. Blood 129, 299-306, 2017.

1. A method for identification of a whole sequence of a variable region of a heavy and/or light chain of one or more isotypes of immunoglobulin in a biological sample, comprising the following steps:
i) extracting intact RNA from said biological sample;
ii) conducting reverse transcription of RNA obtained in step i) and circularization of ds cDNA thus obtained;
iii) conducting two-step reverse PCR with high-fidelity DNA polymerase for amplification with primer pairs directed against a constant region of the circularized ds cDNA transcribed by genes of said one or more immunoglobulin light and/or heavy chain isotypes;
iv) sequencing of DNA single molecules in real time in order to obtain a complete sequence of the variable region of one or more isotypes of the heavy and/or light chains of the immunoglobulins present in the biological sample.

2. The method according to claim 1, further comprising a step v) for classification of the isotypes identified in step iv) based on their relative quantity.

3. The method according to claim 1, wherein said isotypes are α, γ, μ, δ, ε, κ and λ.

4. The method according to claim 1, wherein said immunoglobulins are clonal, namely cancerous.

5. The method according to claim 1, wherein said biological sample derives from a patient affected by monoclonal gammopathy.

6. The method according to claim 5, wherein said monoclonal gammopathy is selected from the group consisting of multiple myeloma, Waldenström's macroglobulinemia, and monoclonal gammopathy of clinical significance (MGCS) or undetermined significance (MGUS).

7. The method according to claim 1, wherein said primer pairs of step iii) directed against the constant region of circularized double-stranded cDNA transcribed by the genes of said one or more isotypes of the light and/or heavy chain of immunoglobulin are m selected from the group consisting of:

| | |
|---|---|
| IGκ-fwd | /5AmMC6/GCAGTCGAACATGTAGCTGACTCAGGTCA |
| universal | CTGCTCATCAGATGGCGGGAA (SEQ ID NO: 1) |
| IGκ-rev | /5AmMC6/TGGATCACTTGTGCAAGCATCACATCGTA |
| universal | GAAGAGCTTCAACAGGGGAGA (SEQ ID NO: 2) |
| IGλ-fwd | /5AmMC6/GCAGTCGAACATGTAGCTGACTCAGGTCA |
| universal | CAGTGTGGCCTTGTTGGCTTG (SEQ ID NO: 3) |
| IGλ-rev | /5AmMC6/TGGATCACTTGTGCAAGCATCACATCGTA |
| universal | GGTCACGCATGAAGGGAGCAC (SEQ ID NO: 4) |
| IGγ-fwd | /5AmMC6/GCAGTCGAACATGTAGCTGACTCAGGTCA |
| universal | CACCGGTTCGGGGAAGTAGTC (SEQ ID NO: 5) |
| IGγ-rev | /5AmMC6/TGGATCACTTGTGCAAGCATCACATCGTA |
| universal | GCTTCTCATGCTCCGTGATGC (SEQ ID NO: 6) |
| IGα-fwd | /5AmMC6/GCAGTCGAACATGTAGCTGACTCAGGTCA |
| universal | CGGCTCCTGGGGGAAGAAGCCC (SEQ ID NO: 7) |
| IGα-rev | /5AmMC6/TGGATCACTTGTGCAAGCATCACATCGTA |
| universal | GCCTTCTCCTGCATGGTGGGCC (SEQ ID NO: 8) |

8. The method according to claim 1, wherein said biological sample is peripheral blood.

9. The method according to claim 1, further comprising a verification step vi) wherein a list of immunoglobulin heavy and/or light chains obtained from the analysis according to steps i)-v) is used for a mapping of proteolytic peptides from serum and/or urine proteins in a urine sample.
